(19) United States
(12) Patent Application Publication
(10) Pub. No.: US 2006/0189804 A1
(43) Pub. Date: Aug. 24, 2006

(54) Synthetic method for the preparation of quinazolin-4-one derivative

(76) Inventor: Andrew Aydon Godfrey, Macclesfield (GB)

Correspondence Address: NIXON & VANDERHYE, PC, 901 NORTH GLEBE ROAD, 11TH FLOOR, ARLINGTON, VA, 22203, US

(21) Appl. No.: 10/562,112

(22) PCT Filed: Jul. 20, 2004
(86) PCT No.: PCT/GB04/03141

§ 371 Date: Feb. 8, 2006

(30) Foreign Application Priority Data
Jul. 28, 2003 (GB) 0317631.0

(51) Int. Cl. C07D 403/02; C07D 239/88

(52) U.S. Cl. 544/284; 544/287

(57) ABSTRACT

A quinazolin-4-one derivative of formula (I), may be made by a process including the step of cyclization an amide of formula (II), to form a quinazolin-4-one derivative of formula (III).

This invention relates to a process for the preparation of certain quinazolin-4-one derivatives which are intermediates in the preparation of further substituted quinazolin-4-one derivatives which possess anti-cancer activity.

Substituted quinazolin-4-one derivatives which possess anti-cancer activity are disclosed in EP-A-0239362 (Imperial Chemical Industries plc et al.), which discloses quinazoline compounds of formula:

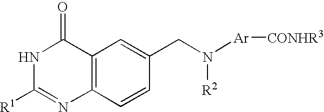

wherein R¹ is alkyl, cycloalkyl, alkenyl, alkynyl, alkoxy or alkylthio each of up to 6 carbon atoms;

aryl, aryloxy or arylalkyl each of up to 10 carbon atoms; halogeno, hydroxy, mercapto, pyridylthio or pyrimidinylthio;

alkyl of up to 3 carbon atoms which bears one, two or three halogeno substituents or which bears one or two substituents selected from hydroxy, amino, pyridylthio, pyrimidinylthio, alkoxy, alkanoyloxy, alkylthio, alkylamino, dialkylamino and alkanoylamino each of up to 6 carbon atoms and aroyloxy and aroylamino each of up to 10 carbon atoms; or

alkoxy of up to 3 carbon atoms which bears one or two substituents selected from hydroxy and alkoxy of up to 6 carbon atoms;

wherein R² is hydrogen, alkyl, alkenyl, alkynyl, hydroxyalkyl, alkoxyalkyl, mercaptoalkyl, alkylthioalkyl, halogenoalkyl, cyanoalkyl, aminoalkyl, alkylaminoalkyl, dialkylaminoalkyl, alkanoylalkyl, carboxyalkyl, carbamoylalkyl or alkanoyl each of up to 6 carbon atoms or aroylalkyl of up to 10 carbon atoms;

wherein Ar is phenylene, naphthylene or heterocyclene which is unsubstituted or which bears one or two substituents selected from halogeno, phenyl, cyano, nitro, hydroxy, amino and carbamoyl and alkyl, alkoxy, halogenoalkyl, alkanoylamino, alkylthio and alkoxycarbonyl each of up to 6 carbon atoms; and wherein R³ is such that R³—NH₂ is an amino acid;

or a pharmaceutically-acceptable salt or ester thereof.

EP-A-0373891 (Imperial Chemical Industries plc el al.), discloses quinazoline compounds of formula:

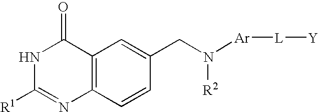

wherein R¹ is hydrogen or amino, or alkyl or alkoxy each of up to 6 carbon atoms;

or R¹ is alkyl of up to 3 carbon atoms which bears a hydroxy substituent, or which bears one, two or three fluoro substituents;

or R¹ is hydroxyalkoxy of up to 3 carbon atoms or alkoxyalkoxy of up to 6 carbon atoms;

wherein the quinazoline ring may bear no further substituents or may bear one further substituent selected from halogeno and from alkyl and alkoxy each of up to 3 carbon atoms;

wherein R² is hydrogen, alkyl, alkenyl, alkynyl, hydroxyalkyl, halogenoalkyl or cyanoalkyl each of up to 6 carbon atoms;

wherein Ar is phenylene or heterocyclene which may be unsubstituted or may bear one or two substituents selected from halogeno, hydroxy, amino and nitro, and from alkyl, alkoxy and halogenoalkyl each of up to 3 carbon atoms;

wherein L is a group of the formula —CO.NH—, —NH.CO—, —CO.NR—, —NR.CO—, —CH═CH—, —CH₂O—, —OCH₂—, —CH₂S—, —SCH₂—, —CO.CH₂—, —CH₂.CO— or —CO.O—, wherein R is alkyl of up to 6 carbon atoms; and

wherein Y is aryl or a hydrogenated derivative thereof each of up to 10 carbon atoms, or heteroaryl or a hydrogenated derivative thereof; or

Y is a group of the formula -A-Y′ in which A is alkylene, cycloalkylene, alkenylene or alkynylene each of up to 6 carbon atoms, and Y′ is aryl or a hydrogenated derivative thereof each of up to 10 carbon atoms, or heteroaryl or a hydrogenated derivative thereof; wherein one constituent methylene group in A may be replaced by an oxy, thio, sulfinyl, sulfonyl or imino group or an alkylimino group of up to 6 carbon atoms;

and wherein each of said aryl or heteroaryl groups, or hydrogenated derivatives thereof, may be unsubstituted or may bear up to three substituents selected from hydroxy, oxo, amino, nitro, cyano, carbamoyl, sulfamoyl, carboxy and halogeno, from alkyl, alkylamino, dialkylamino, N-alkylcarbamoyl, N,N-dialkylcarbamoyl, alkoxycarbonyl, alkanoyloxyalkyl, alkylthio, alkylsulfinyl, alkylsulfonyl, alkoxy, halogenoalkyl, hydroxyalkyl, aminoalkyl, alkylaminoalkyl, dialkylaminoalkyl, carboxyalkyl, alkoxycarbonylalkyl, carbamoylalkyl, N-alkylcarbamoylalkyl and N,N-dialkylcarbamoylalkyl each of up to 6 carbon atoms and from phenyl, pyridyl and phenylalkyl of up to 10 carbon atoms, and wherein each of said phenyl or phenylalkyl groups may bear a substituent selected from halogeno and nitro, and from alkyl and alkoxy each of up to 3 carbon atoms;

or a pharmaceutically-acceptable salt thereof;

provided that when R is hydrogen or amino, or alkyl of up to 6 carbon atoms, and L is a group of the formula —CONH—, then Y is not tetrazolyl.

EP-A-0459730 (Imperial Chemical Industries plc et al.), discloses quinazoline compounds of formula:

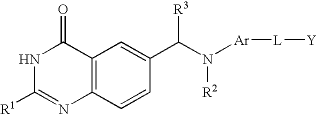

wherein R¹ is hydrogen or amino, or alkyl or alkoxy each of up to 4 carbon atoms;

or R¹ is alkyl of up to 3 carbon atoms which bears a hydroxy substituent, or which bears one, two or three fluoro substituents;

or R¹ is hydroxyalkoxy of up to 3 carbon atoms or alkoxyalkoxy of up to 4 carbon atoms;

wherein the quinazoline ring may bear no further substituent or may bear one further substituent selected from halogeno and from alkyl and alkoxy each of up to 3 carbon atoms;

wherein R² is hydrogen, alkyl, alkenyl, alkynyl, hydroxyalkyl, halogenoalkyl or cyanoalkyl each of up to 4 carbon atoms;

wherein R³ is hydrogen or alkyl of up to 3 carbon atoms;

wherein Ar is phenylene or heterocyclene which may be unsubstituted or may bear one or two substituents selected from halogeno, hydroxy, amino and nitro, and from alkyl, alkoxy and halogenoalkyl each of up to 3 carbon atoms;

wherein L is a group of the formula —CO.NH—, —NH.CO—, —CO.NR⁴—, —NR⁴.CO—, —CH═CH— or —CO.O—, wherein R⁴ is alkyl of up to 4 carbon atoms;

and wherein Y is a group of the formula:

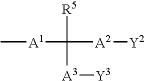

in which R⁵ is hydrogen or alkyl of up to 3 carbon atoms;

A¹ is a direct link or is alkylene of up to 4 carbon atoms, A² is a direct link to Y² or is alkylene of up to 4 carbon atoms, A³ is a direct link to Y³ or is alkylene of up to 4 carbon atoms wherein optionally a constituent methylene group is replaced by an oxy, thio, sulfinyl, sulfonyl, imino or hydroxymethylene group;

Y² is hydroxy, amino, cyano, halogeno or trifluoroacetyl, or alkoxy, alkylamino, dialkylamino, halogenoalkyl, alkylthio, alkylsulfinyl, alkylsulfonyl, alkanoyloxy, alkanoyl or hydroxyalkanoyl each of up to 4 carbon atoms, or aryl, arylthio, arylsulfinyl or arylsulfonyl each of up to 10 carbon atoms, or heteroaryl, heteroarylthio, heteroarylsulfinyl or heteroarylsulfonyl;

and Y³ has any of the meanings defined above for Y², or in addition Y³ is sulfo, N-hydroxycarbamoyl, N-cyanocarbamoyl, carbazoyl or sulfamoyl, or N-alkyl-sulfamoyl, N,N-dialkylsulfamoyl, N-acylsulfamoyl, N-alkylcarbamoyl, N,N-4-dialkylcarbamoyl, N-alkylcarbamoyloxy, N,N-dialkylcarbamoyloxy or N-alkylsulfonylcarbamoyl each of up to 4 carbon atoms, N-phenylsulfonylcarbamoyl or 5-tetrazolyl;

and wherein each of said aryl, arylthio, arylsulfinyl, arylsulfonyl, heteroaryl, heteroarylthio, heteroarylsulfinyl or heteroarylsulfonyl groups may be unsubstituted or may bear one or two substituents selected from hydroxy, oxo, thioxo, amino, nitro, cyano, carbamoyl and halogeno, from alkyl, N-alkylcarbamoyl, N,N-dialkylcarbamoyl, alkylthio, alkylsulfinyl, alkylsulfonyl, alkoxy and halogenoalkyl each of up to 4 carbon atoms, and from phenyl and phenylalkyl of up to 10 carbon atoms;

and wherein said phenyl and phenylalkyl substituents or said N-phenylsulfonylcarbamoyl group may bear a substituent selected from nitro, cyano and halogeno and from alkyl and alkoxy each of up to 3 carbon atoms;

or a pharmaceutically-acceptable salt thereof;

provided that, in the group of the formula -L-Y, no constituent methylene or methine group is attached to more than one heteroatom which is not in a heteroaryl ring.

EP-A-0509643 (Imperial Chemical Industries plc et al.), discloses quinazoline compounds of formula:

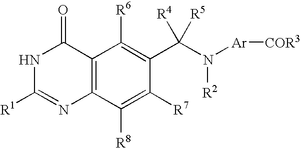

wherein R¹ is hydrogen or amino;

or R¹ is alkyl, alkoxy or alkylthio each of up to 6 carbon atoms;

or R¹ is aryl or aryloxy, each of up to 10 carbon atoms;

or R¹ is halogeno, hydroxy or mercapto;

or R¹ is alkyl of up to 3 carbon atoms which bears one or more substituents selected from halogeno, hydroxy and alkanoylamino each of up to 6 carbon atoms;

or R¹ is alkoxy of up to 3 carbon atoms which bears one or more substituents selected from hydroxy and alkoxy of up to 6 carbon atoms;

wherein R² is hydrogen or alkyl, alkenyl, alkynyl, hydroxyalkyl, alkoxyalkyl, mercaptoalkyl, alkylthioalkyl, halogenoalkyl, cyanoalkyl, aminoalkyl, alkylaminoalkyl, dialkylaminoalkyl, alkanoylalkyl, carboxyalkyl, carbamoylalkyl or alkanoyl each of up to 6 carbon atoms;

wherein Ar is phenylene or heterocyclene which is unsubstituted or which bears one or more substituents selected from halogeno, cyano, nitro, hydroxy, amino and carbamoyl and alkyl, alkoxy, halogenoalkyl, alkanoylamino and alkoxycarbonyl each of up to 6 carbon atoms;

R³ is the residue of a dipeptide in which the first, N-terminal amino acid residue thereof attached to the carbonyl group of COR³ is an L- or D-amino acid residue —NHCH(CO₂H)-A-CO— in which A is an alkylene group of up to 6 carbon atoms and the second amino acid residue is of an alpha-amino acid which has the D-configuration at its asymmetric alpha-carbon atom;

wherein R⁴ is hydrogen or alkyl of up to 4 carbon atoms;

wherein R⁵ is hydrogen or alkyl of up to 4 carbon atoms; and

wherein each of R⁶, R⁷ and R⁸ is hydrogen or alkyl or alkoxy each of up to 4 carbon atoms; or is halogeno;

the quinazoline optionally being in the form of a pharmaceutically-acceptable salt, ester or amide thereof.

EP-A-0562734 (Zeneca Limited et al.), discloses quinazoline compounds of formula:

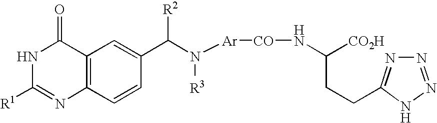

wherein R¹ is (1-4C)alkyl;

the quinazoline ring may optionally bear (at one or two of the 5-, 7- and 8-positions) one or two further substituents selected from halogeno, (1-4C)alkyl and (1-4C)alkoxy;

R² is hydrogen or (1-4C)alkyl;

R³ is hydrogen, (1-4C)alkyl, (3-4C)alkenyl, (3-4C)alkynyl, hydroxy-(2-4C)alkyl, halogeno-(2-4C)alkyl or cyano-(1-4C)alkyl;

and Ar is phenylene or heterocyclene which may optionally bear one or two substituents selected from halogeno, (1-4C)alkyl and (1-4C)alkoxy;

or a pharmaceutically-acceptable salt or ester thereof.

The above patent documents report the synthesis of the compounds in question. Typically the compounds are conceptually broken down via retrosynthetic analysis into key fragments when designing a synthetic route. Thus for example the compound reported as ZD9331 (also known as BGC 9331): 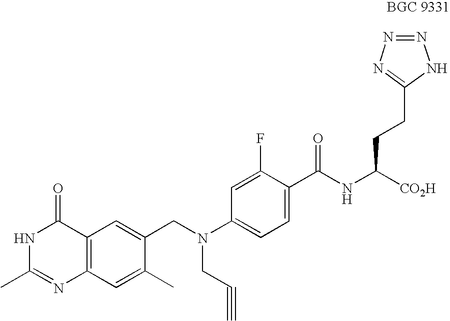
disclosed in EP-A-0562734 may be broken down retrosynthetically as follows: 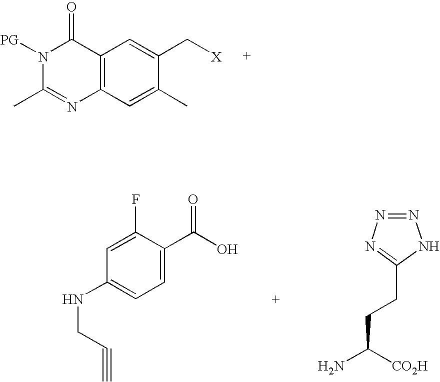
where PG is a protecting group such as pivaloyloxymethyl and X is a leaving group such as Br. The quinazoline component my thus be the compound [6-(bromomethyl)-2,7-dimethyl-4-oxoquinazolin-3(4H)-yl]methyl pivalate:

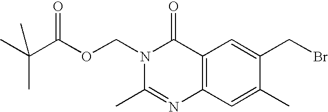

Reported syntheses, for example as disclosed in J. Med. Chem. 1995, 38(6), 994-1004 (Marsham et al.) and J. Med. Chem. 1996, 39(1), 7385 (Bavetsias et al.) make this compound by a scheme including the final free radical bromination step: 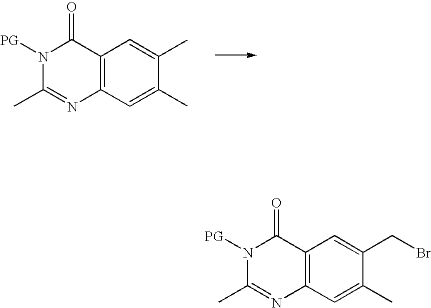
where PG is a protecting group. The method gives a mixture of bromomethyl intermediates and the present inventors have found that this gives poor regioselectivity, only the first product being desired. Typically the following contaminants are found:

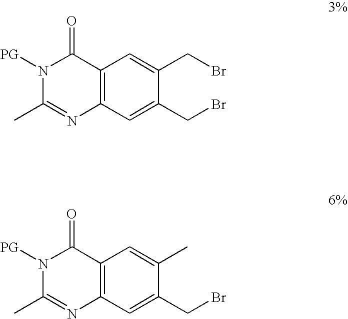

Similarly, a route to the compound raltitrexed: 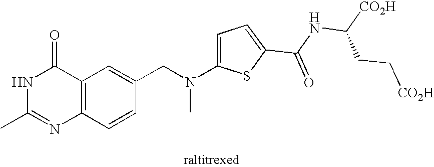
disclosed in EP-A-0239362 would be made by a scheme including the free radical bromination step: 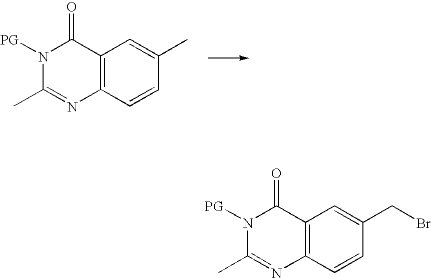
where PG is a protecting group. The method again gives a mixture of bromomethyl intermediates and poor regioselectivity. Typically the following contaminants are found:

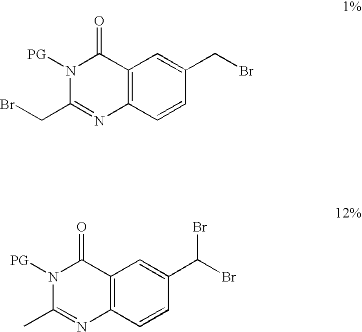

We have now developed an improved route to these key intermediate in which the regiochemistry is defined before cyclization occurs. Accordingly the present invention comprises a process for the preparation of a quinazolin-4-one derivative of formula (I):

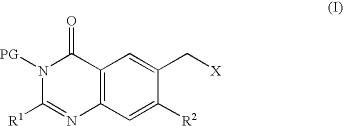

where R¹ and R² are each independently hydrogen or methyl, PG is a protecting group such as pivaloyloxyrnethyl and X is a leaving group such as Br;

including the step of cyclization an amide of formula (II):

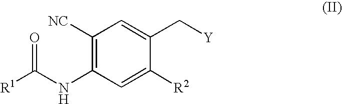

wherein R¹ and R² are as defined above and Y is a leaving group such as OAc;

or a protected derivative thereof;

to form a quinazolin-4-one derivative of formula (III):

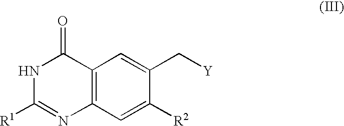

or a protected derivative thereof.

The protecting group PG could be any suitable group for protecting amines, as discussed in “Protective groups in organic synthesis” 3^{rd} Ed, Theodora W Greene and Peter G Wuts, published by John Wiley, ISBN 0-471-16019-9. For example, as well as pivaloyloxymethyl mentioned above, PG could represent BOC (tert-butoxy-carbonyl).

X can represent any suitable leaving group, for example bromide, chloride, iodide, tosylate, mesylate or triflate. Bromide is preferred as it gives the same quinazolin-4-one derivative of formula (I): as has been reported in previous routes, thus removing complications of new related substances in the final bulk drug product.

Y can also represent any suitable leaving group displaceable by X, for example an acyloxy group such as C₁₋₄ acyloxy group or benzoyloxy.

Although the route may use protected derivatives of the amide of formula (II) and quinazolin-4-one derivative of formula (III), additional protection could make the route less efficient and reduce the advantage of the approach. Thus we prefer that the route is done using the step of cyclization an amide of formula (II) to form a quinazolin-4-one derivative of formula (III) without further protection until after the step is completed.

The compound of formula (III) may then be converted into the compound of formula (I) by protection of the ring nitrogen and interconversion of the leaving group Y to X. For example, if X is Br and Y is OAc, hydrogen bromide in acetic acid may be used to effect the conversion.

The cyclization step may be performed under standard conditions. For example, hydrogen chloride in propan-2-ol may be used.

The amide of formula (II) may be made by reacting a compound of formula (IV): 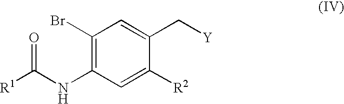
with a cyanide reagent. The compound of formula (IV) is made by a regioselective bromination step from a compound of formula (V) using the reaction step:

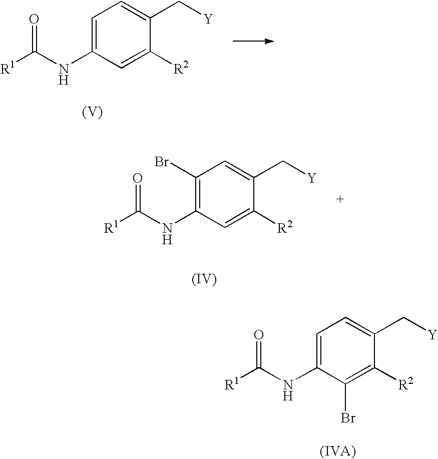

We have found this to be highly regioselective, typically giving an 84:16 mixture in favour of the desired compound of formula (IV). The undesired compound of formula (IVA) is typically lost in the work-up procedure.

Thus in a further aspect of the invention there is provided a process for the preparation of a quinazolin-4-one derivative of formula (I) including the step of brominating a compound of formula (V):

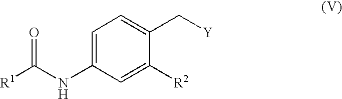

wherein R¹ and R² are as defined above and Y is a leaving group such as OAc;

or a protected derivative thereof;

to form a compound of formula (IV)

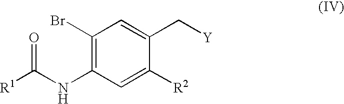

or a protected derivative thereof.

The compound of formula (V) may be made by derivatization of an alcohol of formula (VI):

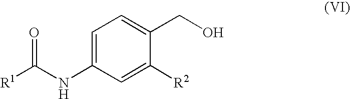

The derivatization and bromination steps may be combined without isolation of the compound of formula (V). The alcohol of formula (VI) may be made by known methods by a scheme such as follows:

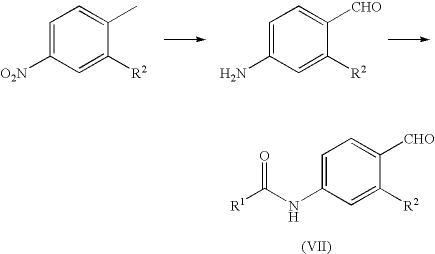

followed by reduction of the aldehyde of formula (VII).

Preferably at least one of R¹ and R² is methyl. Preferably R¹ and R² are both methyl.

In this specification the terms “alkyl”, “alkenyl”, “alkynyl” and “alkylene” include both straight and branched chain groups but references to individual alkyl or alkylene groups, such as “propyl”, are specific for the straight chain group only. An analogous convention applies to other generic terms such as “acyloxy”.

It is to be understood that all the quinazolin-4-one derivatives disclosed may exhibit the phenomenon of tautomerism and that the formulae shown in this specification represent only one of the possible tautomeric forms. It is to be understood therefore that the invention is not limited merely to any one tautomeric form which is illustrated. For example, the quinazolin-4-one derivative of formula (III) may also exist as a quinazolin-4-ol derivative of formula (IIIA):

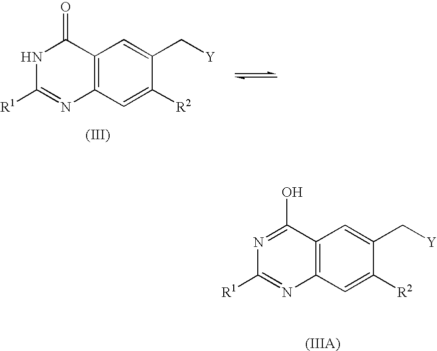

The compound of formula (III) and its halogeno and cyano precursors are key intermediates in the preferred ring-closing process. Thus in a further. aspect of the invention there is provided a quinazolin-4-one derivative of formula (III):

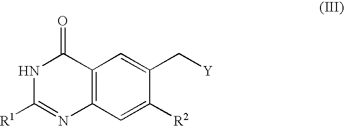

where R¹ and R² are each independently hydrogen or methyl, and Y is a C₁₋₄ acyloxy group or benzoyloxy.

These may be contrasted with intermediates disclosed in the prior art, e.g.:

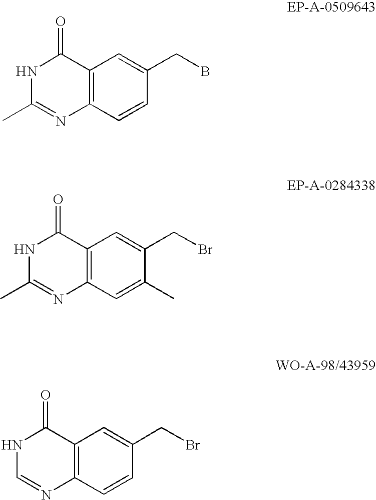

In a further aspect of the invention there is provided an amide of formula (VIII):

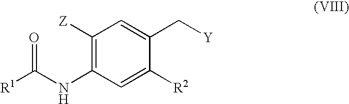

wherein R¹ and R² are each independently hydrogen or methyl, Y is a C₁₋₄ acyloxy group or benzoyloxy and Z is Br or CN.

These may be contrasted with intermediates disclosed in the prior art, e.g. Pharmazie (1969), 24(2), 87-94 (Kleinschmidt et al):

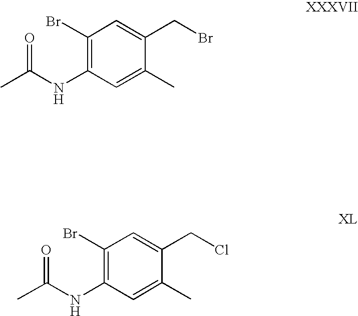

The present invention may be used to prepare any of the relevant compounds in the prior art documents discussed above. For example, it can be used to prepare a quinazoline-4-one of formula (IX):

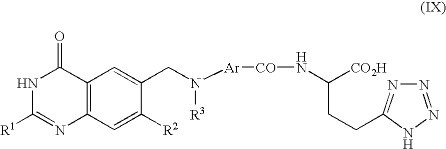

wherein R¹ and R² are each independently hydrogen or methyl;

R³ hydrogen, C₁₋₄ alkyl, C₃₋₄ alkenyl, C₃₋₄ alkynyl, C₂₋₄ hydroxyalkyl C₂₋₄ halogenoalkyl or C₁₋₄ cyanoalkyl;

and Ar is phenylene, thiophenediyl, thiazolediyl, pyridinediyl or pyrimidinediyl which may optionally bear one or two substituents selected from halogeno, hydroxy, amino, nitro, cyano, trifluoromethyl, C₁₋₄ alkyl and C₁₋₄ alkoxy;

or a pharmaceutically-acceptable salt or ester thereof.

It can equally be used to prepare a quinazoline-4-one of formula (X):

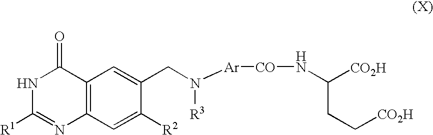

wherein R¹, R², R³ And Ar are as defined above;

or a pharmaceutically-acceptable salt or ester thereof.

A suitable value for R³ when it is C₁₋₄ alkyl, or for a C₁₋₄ alkyl substituent which may be present on Ar, is, for example, methyl, ethyl, propyl or isopropyl.

A suitable value for R³ when it is C₂₋₄ hydroxyalkyl is, for example, 2-hydroxyethyl or 3-hydroxypropyl; when it is C₂₋₄ halogenoalkyl is, for example, 2-fluoroethyl, 2-chloroethyl, 2-bromoethyl, 3-fluoropropyl, 3-chloropropyl or 3-bromopropyl; and when it is C₁₋₄ cyanoalkyl is, for example, cyanomethyl, 2-cyanoethyl or 3-cyanopropyl.

A suitable value for a C₁₋₄ alkoxy substituent which may be present on Ar is, for example, methoxy, ethoxy, propoxy, isopropoxy or butoxy.

A suitable value for a halogeno substituent which may be present on Ar is, for example, fluoro, chloro or bromo.

A suitable value for R³ when it is C₃₋₄ alkenyl is, for example, prop-2-enyl, but-2-enyl, but-3-enyl or 2-methylprop-2-enyl; and when it is C₃₋₄ alkynyl is, for example, prop-2-ynyl or but-3-ynyl.

A suitable value for Ar when it is phenylene is, for example, 1,3- or 1,4-phenylene, especially 1,4-phenylene.

A suitable value for Ar when it is thiophenediyl is, for example, thiophene-2,4-diyl or thiophene-2,5-diyl; when it is thiazolediyl is, for example thiazole-2,4-diyl or thiazole-2,5-diyl; when it is pyridinediyl is, for example, pyridine-2,4-diyl, pyridine-2,5-diyl, pyridine-2,6-diyl or pyridine-3,5-diyl; and when it is pyrimidinediyl is, for example, pyrimidine-2,4-diyl, pyrimidine-2,5-diyl or pyrimidine-4,6-diyl.

As indicated, Ar may carry one or two substituents. A preferred level of substitution in Ar, where substitution is present, is either two substituents or especially one substituent; and the one or two substituents may conveniently be at positions adjacent to the atom bonded to the group —COOH, halogeno substituents such as fluoro being preferred.

Compounds that can be so prepared include the following:

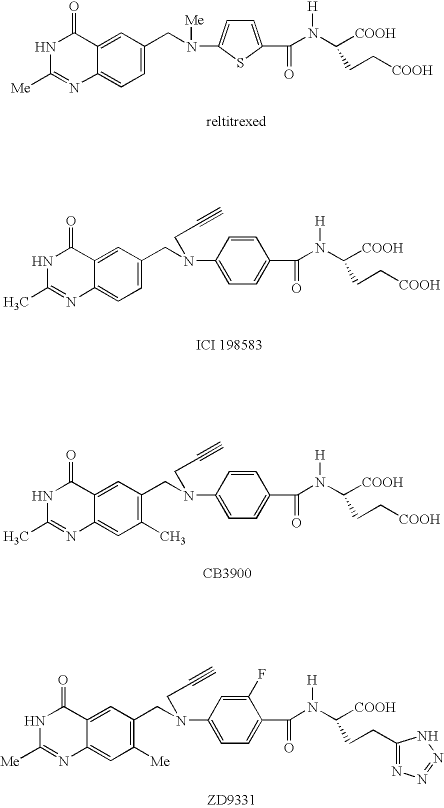

The invention is illustrated by the following Examples.

### EXAMPLE 1

### Synthesis of [6-(bromomethyl)-2,7-dimethyl-4-oxoquinazolin-3(4H)-yl]methyl pivalate

Synthesis was conducted as in Scheme 1.

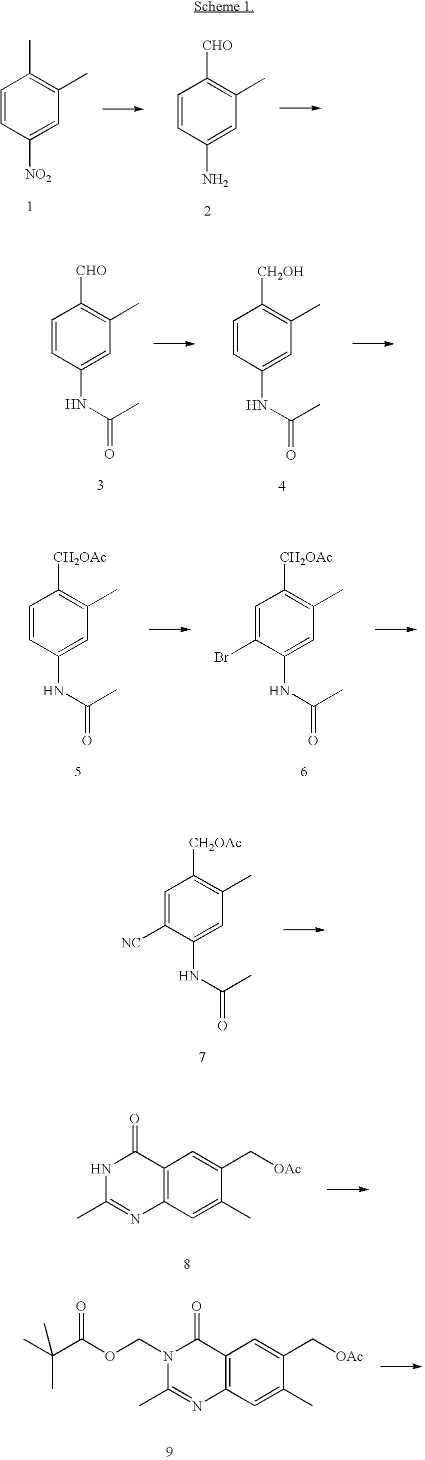

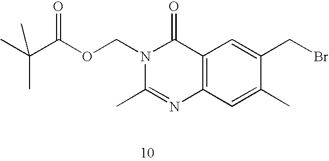

### 6-(Bromomethyl)-2,7-dimethyl-4-oxoquinazolin-3(4H)-yl]methyl pivalate

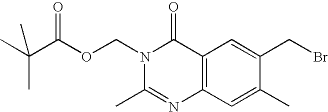

Hydrogen bromide in acetic acid (30% w/w 885 g, 3.28 mol) was added in one portion to a slurry of [6-[(acetyloxy)methyl]-2,7-dimethyl-4-oxoquinazolin-3(4H)-yl]methyl pivalate (1.182 kg, 3.28 mol) in acetic acid (5.9 litres). The solution was heated to 60° C., and hydrogen bromide in acetic acid (1.327 kg, 4.92 mol) was added over two hours. After a further three hours at 60° C. [6-(bromomethyl)-2,7-dimethyl-4-oxoquinazolin-3(4H)-yl]methyl pivalate hydrobromide was crystallised out of solution by cooling to 16° C. and holding for eighteen hours. After isolation and washing sequentially with acetic acid then toluene [6-(bromomethyl)-2,7-dimethyl-4-oxoquinazolin-3(4H)-yl]methyl pivalate hydrobromide was dried to constant weight at 50° C. in vacuo.

[6-(Bromomethyl)-2,7-dimethyl-4-oxoquinazolin-3(4H)-yl]methyl pivalate hydrobromide (10)1.349 kg was isolated representing a yield of 89%.

¹H NMR δ (DMSO-d6): 1.3 (s, 9H), 2.6 (s, 3H), 2.75 (s, 3H), 5.0 (s, 2H), 6.2 (s, 2H), 7.7 (s, 1H), 8.3 (s, 1H).

MS m/z 380 (M⁺)

### 6-[(Acetyloxy)methyl]-2,7-dimethyl-4-oxoquinazolin-3(4H)-yl]methyl pivalate

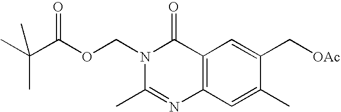

Potassium carbonate (2.234 kg, 14.22 mole) was charged in one portion to a solution of (2,7-dimethyl-4-oxo-3,4-dihydroquinazolin-6-yl)methyl acetate hydrochloride (1.50 kg, 5.29 mole) in dimethyl sulfoxide (15 litres) at 50° C. After holding for sixteen hours at 50° C. chloromethyl pivalate (1.027 kg, 6.61 mole) was added over 2.5 hours. After holding at 50° C. for a further thirty minutes the mixture was drowned out into water (25.0 litres). [6-[(acetyloxy)methyl]-2,7-dimethyl-4-oxoquinazolin-3(4H)-yl]methyl pivalate was isolated by filtration and washed with water. O-alkylated product is removed by sequentially washing with propan-2-ol and isohexane prior to drying at ambient temperature in vacuo.

[6-[(acetyloxy)methyl]-2,7-dimethyl-4-oxoquinazolin-3(4H)-yl]methyl pivalate (9) 1.18 kg was isolated representing a yield of 62%.

¹ H NMR δ (DMSO-d6): 1.2 (s, 9H), 2.1 (s, 3H), 2.4 (s, 3H), 2.6 (s, 3H), 5.2 (s, 2H), 6.1 (s, 2H), 7.5 (s, 1H), 8.1 (s, 1H).

### (2,7-Dimethyl-4-oxo-3,4-dihydroquinazolin-6-yl)methyl acetate hydrochloride

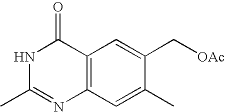

Hydrogen chloride gas (0.12 kg, 3.29 mole) was added over sixty minutes, to a slurry of N-[4-(acetyloxy)-2-cyano-5-methylphenyl]-acetamide (0.67 kg, 2.7 mole) in propan-2-ol (6.7 litre). On cooling to 30° C. (2,7-dimethyl-4-oxo-3,4-dihydroquinazolin-6-yl)methyl acetate hydrochloride crystallised out of solution. The product was isolated by filtration, washed with propan-2-ol and dried to constant weight at 50° C. in vacuo.

(2,7-dimethyl-4-oxo-3,4-dihydroquinazolin-6-yl)methyl acetate hydrochloride (8) 0.662 kg was isolated representing a yield of 87%.

¹H NMR δ (DMSO-d6): 2.1 (s, 3H), 2.4 (s, 3H), 2.7 (s, 3H), 5.2 (s, 2H), 7.7 (s, 1H), 8.1 (s, 1H).

### N-[4-(Acetyloxy)-2-cyano-5-methylphenyl]-acetamide

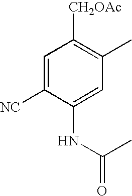

4-(acetylamino)-5-bromo-2-methylphenyl acetate (50 g, 0.167 mole), copper(i) cyanide (14.2 g, 0.159 mole) and dimethylformamide (100 ml) were heated at 90° C. under an atmosphere of nitrogen. After six hours the mixture was cooled to 60° C. and treated portionwise with zinc powder (13.1 g, 0.2 mole), the slurry was reheated to 90° C., screened through Celite, cooled to 50° C. and diluted with water (400 ml). On cooling to 20° C. the product was isolated by filtration, washed with water, and dried to constant weight at 50° C. in vacuo.

N-[4-(acetyloxy)-2-cyano-5-methylphenyl]acetamide 41.4 g was isolated representing a yield of 84%.

¹H NMR δ (DMSO-d6): 2.1 (s, 3H), 2.25 (s, 3H), 2.5 (s, 3H), 5.3 (s, 2H), 7.6 (s, 1H), 7.9 (s, 1H), 10.3 (s, 1H)

### 4-(Acetylamino)-2-methylphenyl acetate and 4-(acetylamino)-5-bromo-2-methylphenyl acetate

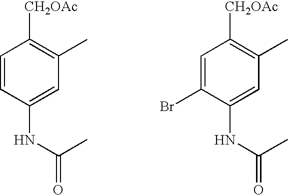

Telescoped reaction avoiding the isolation of 4-(acetylamino)-2-methylphenyl acetate (5).

Triethylamine (63 ml, 0.45 mole) was added in one portion to a slurry of N-[4-(hydroxymethyl)-3-methylphenyl]acetamide (54 g, 0.3 mole), in ethyl acetate (540 ml) at ambient temperature. The slurry was heated to 50° C., acetyl chloride (30 ml, 0.42 mole) was added over two hours, after a further thirty minutes the mixture was cooled to 20° C. The slurry was extracted sequentially with water (2×270 ml) and saturated brine (270 ml). The ethyl acetate extract was solvent swapped into acetonitrile by distillation. The acetonitrile solution of 4-(acetylamino)-2-methylphenyl acetate is treated with a solution of 1,3-dibromo-5,5-dimethylhydantoin (Bromodan) (48.6 g, 0.17 mole) in acetonitrile (380 ml) at 50° C., after 60 minutes the reaction mixture was cooled to 20° C. and drowned out into water (1350 ml). 4-(acetylamino)-5-bromo-2-methylphenyl acetate was isolated by filtration, washed with water and dried to constant weight at 50° C. in vacuo. The regioisomer 4-(acetylamino)-3-bromo-2-methylphenyl acetate was lost to the aqueous acetonitrile wash. 4-(acetylamino)-5-bromo-2-methylphenyl acetate 56 g was isolated representing a yield of 62%.

¹H NMR δ (DMSO-d6): 2.1 (s, 3H), 2.2 (s, 3H), 2.3 (s, 3H), 5.0 (s, 2H), 7.6 (s, 1H) 7.4 (s, 1H), 9.5 (s, 1H).

### N-[4-(Hydroxymethyl)-3-methylphenyl]acetamide

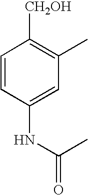

Prepared according to EP-A-0268989 (Fujisawa Pharmaceutical Co. Ltd.).

### EXAMPLE 2

### Synthesis of BGC 9331

Synthesis was conducted as in Scheme 2.

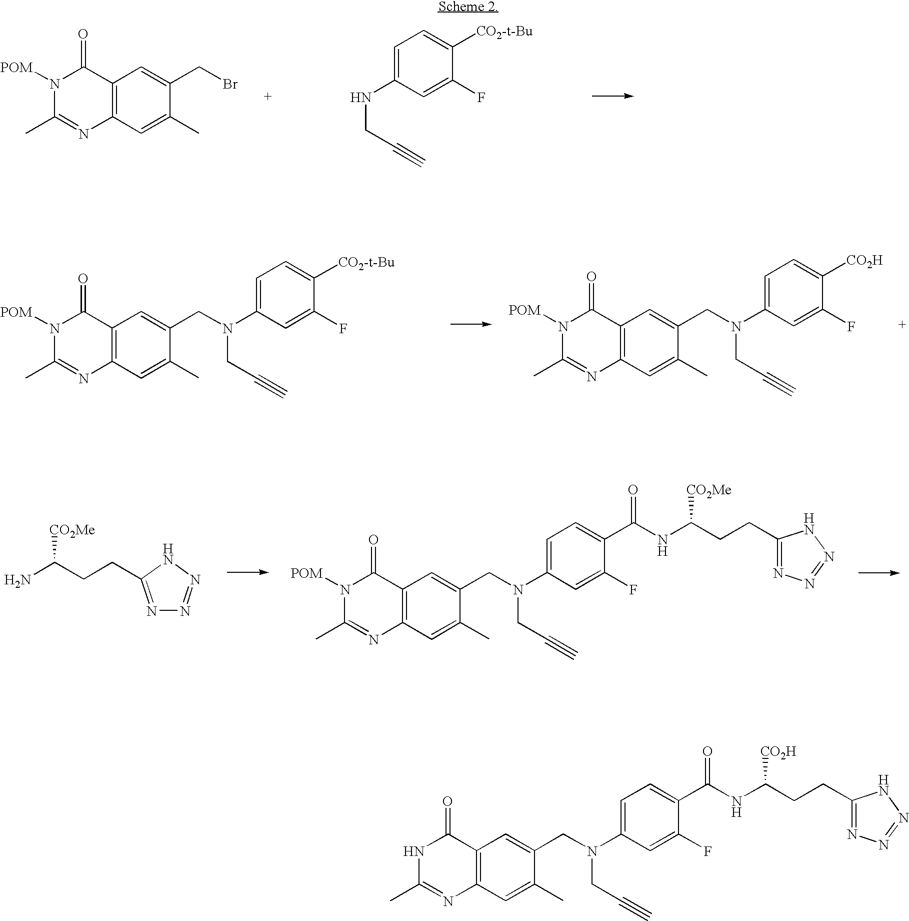

### (2S)-2-({4-[[(2,7-Dimethyl-4-oxo-3,4-dihydroquinazolin-6-yl)methyl](prop-2-yn-1-yl)amino]-2-fluorobenzoyl}amino)-4-(1H-tetrazol-5-yl)butanoic acid

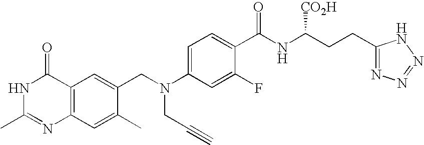

Aqueous sodium hydroxide (48% w/w, 12 litres, 211.5 mole) diluted with water (122 litre) was added to a stirred solution of methyl (2S)-2-({4-[[(3-{[(2,2-dimethylpropanoyl)oxy]methyl}-2,7-dimethyl-4-oxo-3,4-dihydroquinazolin-6-yl)-methyl](prop-2-yn-1-yl)amino]-2-fluorobenzoyl}amino)-4-(1H-tetrazol-5-yl)-butanoate (34.75 kg, 52.6 mole), tetrahydrofuran (348 litres), and water (122 litre) at 15° C. The solution was heated to 24° C. and held at this temperature for 19 hours. Water (35 litre) and sodium bisulfite (8.1 kg, 77.8 mole) were charged sequentially to the reaction mixture, after stirring for 40 minutes the contents were allowed to settle, the upper tetrahydrofuran phase was removed and discarded. The lower aqueous phase was diluted with water (54 litres) and tetrahydrofuran (446 litre) then heated to 40° C. 2.8 M Sulfuric acid (35 litre) was added below 50° C., the contents were allowed to settle and the lower acidic aqueous phase was discarded. (2S)-2-({4-[[(2,7-dimethyl-4-oxo-3,4-dihydroquinazolin-6-yl)methyl](prop-2-yn-1-yl)amino]-2-fluoro-benzoyl}amino)-4-(1H-tetrazol-5-yl)butanoic acid was precipitated by the addition of cyclohexane (175 litre), the precipitate was isolated by filtration, washed sequentially with a mixture of tetrahydrofuran (70 litres)/cyclohexane (35 litres) and finally water (2×209 litres) prior to drying at 50° C. (2S)-2-({4-[[(2,7-dimethyl-4-oxo-3,4-dihydroquinazolin-6-yl)methyl](prop-2-yn-1-yl)amino]-2-fluorobenzoyl}amino)-4-(1H-tetrazol-5-yl)butanoic acid 25.65 kg was isolated representing a yield of 92%.

### Methyl (2S)-2-({4-[[(3-{[(2,2-dimethylpropanoyl)oxy]methyl}-2,7-dimethyl-4-oxo-3,4-dihydroquinazolin-6-yl)methyl](prop-2-yn-1-yl)amino]-2-fluorobenzoyl}amino)-4-(1H-tetrazol-5-yl)butanoate

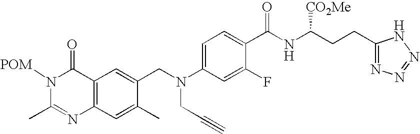

Thionyl chloride (555 ml, 7.61 mole) was added over 30 minutes to a solution of 4-[[(3- {[(2,2-dimethylpropanoyl)oxy]methyl}-2,7-dimethyl-4-oxo-3,4-dihydroquinazolin-6-yl)methyl](prop-2-yn-1-yl)amino]-2-fluorobenzoic, acid (2.681 kg, 5.43 mole) in dichloromethane (26.8 litres), under an atmosphere of nitrogen, at 10° C., after this time the solution was warmed to 20° C. The acid chloride solution was added over 3 hours to a solution of methyl (2S)-2-amino-4-(1H-tetrazol-5-yl)-butanoate (1.214 kg, 5.97 mole), diisopropylethylamine (5.7 litres 32.6 mol) in dichloromethane (5.3 litres), under an atmosphere of nitrogen at 10° C., after a further 16 hours glacial acetic acid (1.46 kg, 24.4 mole) was added. The dichloromethane solution was diluted with methanol (5.4 litres) then washed sequentially with water (2×13 litres), and finally with saturated brine (13.4 litres). Dichloromethane was exchanged for methanol by distillation at atmospheric pressure to achieve a final volume for the methanol solution of 40 litres. Water (12 litres) was added to the methanol solution at 50° C. on cooling to 25° C. methyl (2S)-2-({4 [[(3-{[(2,2-dimethylpropanoyl)oxy]methyl}-2,7-dimethyl-4-oxo-3,4-dihydroquinazolin-6-yl)methyl](prop-2-yn-1-yl)amino]-2-fluorobenzoyl}amino)-4-(1H-tetrazol-5-yl)-butanoate crystallised out of solution. The product was isolated by filtration and washed with a mixture of methanol (3.6 litre)/water (11 litres), prior to drying at 50° C. Methyl (2S)-2-({4-[[(3-{[(2,2-dimethylpropanoyl)oxy]methyl}-2,7-dimethyl-4-oxo-3,4-dihydroquinazolin-6-yl)methyl](prop-2-yn- 1 -yl)amino]-2-fluorobenzoyl}-amino)-4-(1H-tetrazol-5-yl)butanoate (2.94 kg) was isolated representing an 82% yield.

### 4-[[(3-{([2,2-Dimethylpropanoyl)oxy]methyl}-2,7-dimethyl-4-oxo-3,4-dihydroquinazolin-6-yl)methyl](prop-2-yn-1-yl)amino]-2-fluorobenzoic acid

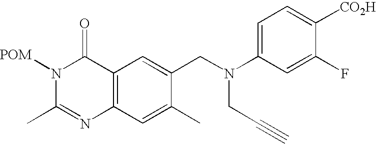

tert-Butyl 4-[[(3-{[(2,2-dimethylpropanoyl)oxy]methyl}-2,7-dimethyl-4-oxo-3,4-dihydroquinazolin-6-yl)methyl](prop-2-yn-1-yl)amino]-2-fluorobenzoate (33.2 kg, 60.47 mole) and formic acid (205 litres) were heated at 40° C. for 5 hours, after this time water (306 litres) was added over 3 hours. 4-[[(3-{[(2,2-dimethylpropanoyl)-oxy]methyl}-2,7-dimethyl-4-oxo-3,4-dihydroquinazolin-6-yl)methyl](prop-2-yn-1-yl)amino]-2-fluorobenzoic acid was isolated by filtration and washed with water (3×69 litres) prior to drying at 50° C. 4-[[(3-{[(2,2-dimethylpropanoyl)oxy]methyl}-2,7-dimethyl-4-oxo-3,4-dihydroquinazolin-6-yl)methyl](prop-2-yn-1-yl)amino]-2-fluorobenzoic acid (29.2 kg) was isolated representing a yield of 98%.

### tert-Butyl 4-[[(3-{[(2,2-Dimethylpropanoyl)oxy]methyl}-2,7-dimethyl-4-oxo-3,4-dihydroquinazolin-6-yl)methyl](prop-2-yn-1-yl)amino]-2-fluorobenzoate

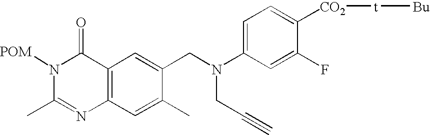

A solution of sodium hydrogen carbonate (0.782 kg, 9.3 mole) in water (13.2 litres) was added over 30 minutes to a slurry of [6-(bromomethyl)-2,7-dimethyl-4-oxoquinazolin-3(4H)-yl]methyl pivalate hydrogen bromide (2.578 kg, 5.58 mole) in toluene (25.0 litres) at 65° C. After 1 hour the lower aqueous phase was removed and discarded. The toluene solution was washed with a further portion of water (13.2 litres), the lower aqueous phase was discarded prior to drying by azeotropic distillation. Distillation was continued until the kettle residue volume was 6 litres, the contents were cooled to 15° C. before adjusting the internal pressure to atmospheric pressure with argon. tert-Butyl 2-fluoro-4-(prop-2-yn-1-ylamino)benzoate (1.324 kg, 5.31 moles) and 2,6-lutidine (0.854 kg, 1.5 moles) were charged to the toluene solution, then the internal temperature was slowly ramped to 105° C. The batch was held at 105° C. for 24 hours before cooling to 65° C. Toluene (7.2 litres), water (13.2 litres) and hydrochloric acid (36% w/w, 0.269 kg, 0.5 mole) were charged sequentially, after stirring for 15 minutes at 65° C. the lower aqueous phase was discarded. The toluene solution was concentrated under reduced pressure, and after adjusting the internal temperature to 75° C. the vacuum was released, cyclohexane (7.9 litres) was charged over 5 minutes. tert-Butyl 4-[[(3-{[(2,2-dimethylpropanoyl)oxy]-methyl}-2,7-dimethyl-4-oxo-3,4-dihydroquinazolin-6-yl)methyl](prop-2-yn-1-yl)-amino]-2-fluorobenzoate crystallised from solution on cooling to 20°C., the product was isolated by filtration and washed with a mixture of toluene (2.66 litres)/cyclohexane (1.43 litres) prior to drying at 50° C. tert-Butyl 4-[[(3-{[(2,2-dimethyl-propanoyl)oxy]methyl}-2,7-dimethyl-4-oxo-3,4-dihydroquinazolin-6-yl)methyl]-(prop-2-yn-1-yl)amino]-2-fluorobenzoate 2.33 kg was isolated representing a yield of 80%.

1. A process for the preparation of a quinazolin-4-one derivative of formula (I): 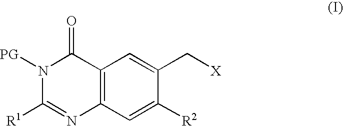
where R¹ and R² are each independently hydrogen or methyl, PG is a protecting group and X is a leaving group;
including the step of cyclization an amide of formula (II): 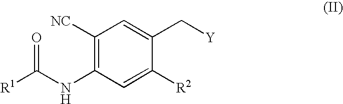
wherein R¹ and R² are as defined above and Y is a leaving group; or a protected derivative thereof;
to form a quinazolin-4-one derivative of formula (III): 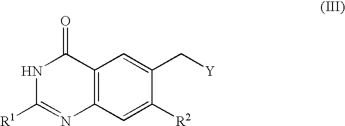
or a protected derivative thereof.

2. A process as claimed in claim 1 wherein the amide of formula (II) is made by reacting a compound of formula (IV): 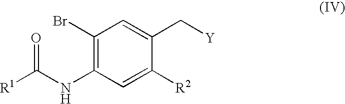
with a cyanide reagent.

3. A process as claimed in claim 2 wherein the compound of formula (IV) is made by a regioselective bromination step from a compound of formula (V) using the reaction step:

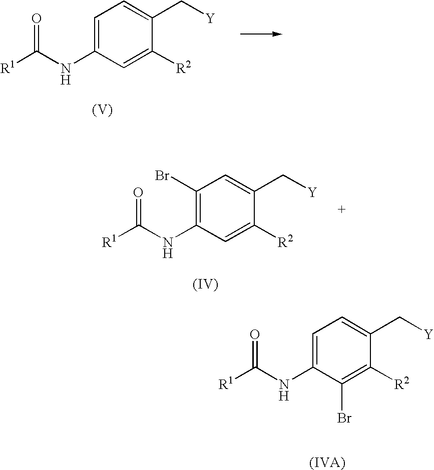

4. A process for the preparation of a quinazolin-4-one derivative of formula (I): 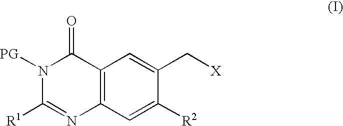
where R¹ and R² are each independently hydrogen or methyl, PG is a protecting group and X is a leaving group;
including the step of brominating a compound of formula (V): 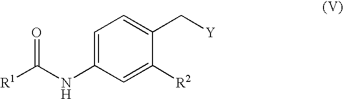
wherein R¹ and R² are as defined above and Y is a leaving group;
or a protected derivative thereof;
to form a compound of formula (IV) 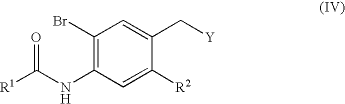
or a protected derivative thereof.

5. A process as claimed in claim 4 wherein the compound of formula (V) is made by derivatization of an alcohol of formula (VI):

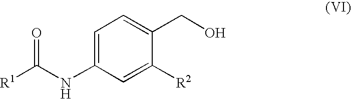

6. A process as claimed in claim 1 wherein at least one of R¹ and R² is methyl.

7. A process as claimed in claim 6 wherein R¹ and R² are both methyl.

8. A quinazolin-4-one derivative of formula (III): 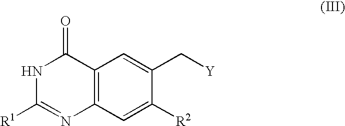
where R¹ and R² are each independently hydrogen or methyl, and Y is a C₁₋₄ acyloxy group or benzoyloxy.

9. An amide of formula (VIII): 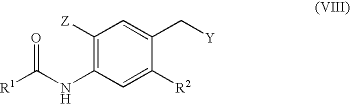
wherein R¹ and R² are each independently hydrogen or methyl, Y is a C₁₋₄ acyloxy group or benzoyloxy and Z is Br or CN.

10. A compound as claimed in claim 8 wherein at least one of R¹ and R² is methyl.

11. A compound as claimed in claim 10 wherein R¹ and R² are both methyl.

12. A process as claimed in claim 1 wherein the process is used to prepare a quinazoline-4-one of formula (IX): 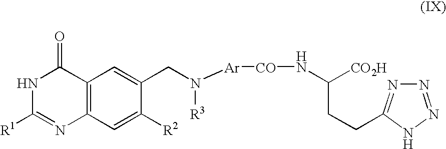
wherein R¹ and R² are each independently hydrogen or methyl;
R³ hydrogen, C₁₋₄ alkyl, C₃₋₄ alkenyl, C₃₋₄ alkynyl, C₂₋₄ hydroxyalkyl C₂₋₄ halogenoalkyl or C₁₋₄ cyanoalkyl;
and Ar is phenylene, thiophenediyl, thiazolediyl, pyridinediyl or pyrimidinediyl which may optionally bear one or two substituents selected from halogeno, hydroxy, amino, nitro, cyano, trifluoromethyl, C₁₋₄ alkyl and C₁₋₄ alkoxy;
or a pharmaceutically-acceptable salt or ester thereof.

13. A process as claimed in claim 1 wherein the process is used to prepare a quinazoline-4-one of formula (X): 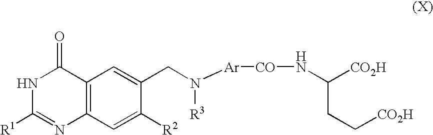
wherein R¹ and R² are each independently hydrogen or methyl;
R³ hydrogen, C₁₋₄ alkyl, C₃₋₄ alkenyl, C₃₋₄ alkynyl, C₂₋₄ hydroxyalkyl C₂₋₄ halogenoalkyl or C₁₋₄ cyanoalkyl;
and Ar is phenylene, thiophenediyl, thiazolediyl, pyridinediyl-or pyrimidinediyl which may optionally bear one or two substituents selected from halogeno, hydroxy, amino, nitro, cyano, trifluoromethyl, C₁₋₄alkyl and C₁₋₄alkoxy;
or a pharmaceutically-acceptable salt or ester thereof.
